(19) United States
(12) Patent Application Publication
(10) Pub. No.: US 2025/0354111 A1
(43) Pub. Date: Nov. 20, 2025

(54) BOVINE SEX-SORTED SPERM SORTING SOLUTION MEDIATED BY SMALL MOLECULE COMPOUND AND ITS APPLICATION

(71) Applicant: Inner Mongolia University, Inner Mongolia (CN)

(72) Inventors: Lei YANG, Inner Mongolia (CN); Dezheng WANG, Inner Mongolia (CN); Guangpeng LI, Inner Mongolia (CN); Anqi DI, Inner Mongolia (CN); Lishuang SONG, Inner Mongolia (CN); Chunling BAI, Inner Mongolia (CN); Xuefei LIU, Inner Mongolia (CN)

(73) Assignee: Inner Mongolia University, Inner Mongolia (CN)

(21) Appl. No.: 19/207,389

(22) Filed: May 14, 2025

(30) Foreign Application Priority Data
May 14, 2024 (CN) 202410592180.0

(51) Int. Cl. C12N 5/071

(52) U.S. Cl. CPC C12N 5/0612

(57) ABSTRACT

The present invention provides a bovine sex-sorted sperm sorting solution mediated by a small molecule compound and its application. The sperm sorting solution comprises a sperm sorting protection solution and a sex control functional component. The sperm sorting protection solution contains sodium chloride, potassium chloride, calcium chloride dihydrate, sodium dihydrogen phosphate, glucose, sodium pyruvate, sodium bicarbonate, penicillin-streptomycin, and phenol red. The sex control functional component includes creatine and a 24e drug. The concentration of creatine is 450 μM-550 μM, and the concentration of the 24e drug is 0.27 μM-0.33 μM. The 24e drug is a pyrido [3,2-d]pyrimidine-based TLR7&8 dual agonist. The present invention can meet the large demand for sex-sorted semen in production. Moreover, the sorting process is carried out under the suitable conditions for sperm and does not require complex treatment of sperm. It takes less time for sorting and causes less damage to sperm.

### CROSS-REFERENCE TO RELATED APPLICATION

This application claims the priority benefit of China application no. 202410592180.0, filed on May 14, 2024. The entirety of each of the above-mentioned patent applications is hereby incorporated by reference herein and made a part of this specification.

### REFERENCE TO A SEQUENCE LISTING

The instant application contains a Sequencing Listing which has been submitted electronically in XML format and is hereby incorporated by reference in its entirety. Said XML copy, created on Mar. 25, 2025, is named 154864_SEQUENCELISTING and is 6,483 bytes in size.

### TECHNICAL FIELD

The present invention relates to the field of animal reproduction and breeding, and particularly provides a bovine sex-sorted sperm sorting solution mediated by a small molecule compound and its application.

### BACKGROUND OF RELATED ART

Sex control technology refers to the technique by which human intervention enables animal breeding to produce offspring of the desired sex. The bovine sex-sorted semen technology is hailed as the third technological revolution for rapid expansion of improved breeds after artificial insemination and embryo transfer. It mainly involves sorting X and Y sperm and using the sorted semen for insemination to produce offspring of the expected sex.

Currently, based on the differences in size, motility, charge, surface antigens, and DNA content of X and Y sperm, sperm sorting methods mainly include density gradient centrifugation, electrophoresis, protein immunological sorting, and flow cytometry sorting. However, density gradient centrifugation, electrophoresis, and protein immunological sorting all have problems such as insufficient theoretical basis, poor reproducibility, and unreliable results. Flow cytometry sorting is currently the most successful method for sorting bull sperm by gender. Nevertheless, flow cytometry sorting still has many drawbacks. For example, the motility of sorted sperm and the percentage of acrosome-intact sperm are significantly decreased, resulting in weakened sperm fertility, lower conception rates after artificial insemination, and higher requirements for insemination techniques. Secondly, the flow cytometers used for sperm sorting are too expensive and have a slow sorting speed, which cannot be obtained in individual farms or laboratories and cannot meet the large demand for sex-sorted semen in production.

### SUMMARY OF THE INVENTION

The objective of the present invention is to provide a bovine sex-sorted sperm sorting solution mediated by a small molecule compound and its application, so as to address the issues associated with the aforementioned separation methods. The specific embodiments are as follows:

A bovine sex-sorted sperm sorting solution mediated by a small molecule compound, including a sperm sorting protection solution and a sex control functional component. The sperm sorting protection solution consists of sodium chloride, potassium chloride, calcium chloride dihydrate, sodium dihydrogen phosphate, glucose, sodium pyruvate, sodium bicarbonate, penicillin-streptomycin, and phenol red.

The sex control functional component includes creatine and a 24e drug.

The concentration of creatine is 450 μM-550 μM.

The concentration of the 24e drug is 0.27 μM-0.33 μM. The 24e drug is a pyrido[3,2-d] pyrimidine-based TLR7&8 dual agonist.

Furthermore, each 1000 ml of the sperm sorting protection solution is prepared by mixing 6.902 g of sodium chloride, 0.300 g of potassium chloride, 0.330 g of calcium chloride dihydrate, 0.097 g of sodium dihydrogen phosphate, 2.500 g of glucose, 0.138 g of sodium pyruvate, 3.105 g of sodium bicarbonate, 2.5 mL of penicillin-streptomycin, 400 μL of phenol red, and deionized water.

Moreover, 98.41 μg to 120.28 μg of the 24e drug and 59.01 mg to 72.12 mg of creatine are added to each 1000 ml of the sperm sorting protection solution.

In addition, the molecular formula of the 24e drug is C₂₁H₂₈N₆, and its molecular structural formula is shown in FIG. 6.

A usage method of the bovine sex-sorted sperm sorting solution mediated by the small molecule compound is as follows:
(1) detecting the vitality of the collected fresh bovine semen, diluting the collected fresh bovine semen with the sperm sorting protection solution, centrifuging, removing the supernatant, and resuspending the sperm with the bovine sex-sorted sperm sorting solution;
(2) incubating statically at 35° C.-40° C. for 60 min-90 min to obtain the incubated layered semen;
(3) separating the above layered semen to obtain the supernatant and the precipitate, where the supernatant is the Y semen and the precipitate is the X semen; and
(4) eluting the drugs in the Y semen and X semen respectively with the sperm sorting protection solution to obtain the Y sex-sorted semen and X sex-sorted semen for any one of in vitro fertilization, artificial insemination, and intracytoplasmic sperm injection.

Furthermore, the primers used for verifying the sorting effect of the bovine sex-sorted sperm sorting solution mediated by the small molecule compound by real-time fluorescence quantitative PCR are respectively a Y chromosome-specific gene SRY gene primer pair, an X chromosome-specific gene PLP gene primer pair, and an internal reference gene GAPDH gene primer pair. The Y chromosome-specific gene SRY gene primer pair is SRY-F and SRY-R. The nucleotide sequence of SRY-F is as shown in SEQ ID NO.1 (5′-GAAAATAAGCACAAGAAAGTCCAGG-3′); the nucleotide sequence of SRY-R is as shown in SEQ ID NO.2 (5′-CAAAAGGAGCATCACAGCAGC-3′).

The X chromosome-specific gene PLP gene primer pair is PLP-F and PLP-R. The nucleotide sequence of PLP-F is as shown in SEQ ID NO.3 (5′-TGTGTGGCTCCAACCTTCTG-3′); the nucleotide sequence of PLP-R is as shown in SEQ ID NO.4 (5′-GCTGTCTCCAAGCTATCCCA-3′).

The internal reference gene GAPDH primer pair is GAPDH-F and GAPDH-R. The nucleotide sequence of GAPDH-F is as shown in SEQ ID NO.5 (5′-AAGAGGCCACTCTGTTACCC-3′); and the nucleotide sequence of GAPDH-R is as shown in SEQ ID NO.6 (5′-TTACTAGAGGGTGGGCAAGC-3′).

On the other hand, the present invention also provides application of the bovine sex-sorted sperm sorting solution mediated by the small molecule compound. It can be used for identifying the sex of bull sperm.

It can also be used for preparing a kit for identifying the sex of bull sperm.

Additionally, it can be used for sorting or screening bull X and Y sperm.

Moreover, it can be used for preparing a kit for sorting or screening bull X and Y sperm.

### Advantageous Effects

1. The method for sorting X and Y sperm using the bovine sex-sorted sperm sorting solution mediated by the small molecule compound provided by the present invention is simple and easy to implement, and can meet the large demand for sex-sorted semen in production. Moreover, the sorting process is carried out under the suitable conditions for sperm and does not require complex treatment of sperm. Therefore, it takes less time for sorting, causes less damage to sperm, does not affect the fertility of sperm, has a higher conception rate after artificial insemination, and has lower requirements for insemination techniques.

2. The activation of the 24e drug provided by the present invention selectively inhibits the motility of X sperm while having no effect on Y sperm, but does not change the viability of sperm or the formation of the acrosome. The difference in sperm motility enables the separation of Y sperm and X sperm. Creatine can promote sperm movement, so that Y sperm floats up faster and the stratification is more obvious, improving the sorting efficiency.

3. Experimental studies have found that TLR7&8 specifically expressed on the surface of X sperm are present in approximately 50% of round spermatids and approximately 50% of epididymal sperm in the testis. Among them, TLR7 is located in the tail, and TLR8 is located in the middle piece. Activation of TLR7 and TLR8 not only inhibits hexokinase activity but also inhibits mitochondrial activity in sperm, thereby weakening the glycolytic pathway and the tricarboxylic acid cycle, and further reducing the production of ATP, resulting in a decrease in the motility of X sperm, thereby achieving the sorting of sex-sorted sperm.

### BRIEF DESCRIPTION OF THE DRAWINGS

FIG. 1 shows the effect of different amounts of the 24e drug on the number of sperm in the upper layer solution in Example 1.

FIG. 2 shows the effect of the working concentration of creatine on the sorting effect of X sperm and Y sperm in Example 2.

FIG. 3A shows the proportion of X sperm and Y sperm in the control group in Method 1 of Example 3, and FIG. 3B is a bar graph of the proportion of X sperm and Y sperm in the control group in Method 1 of Example 3.

FIG. 4A shows the number of Y sperm and X sperm in the supernatant of the experimental group in Method 1 of Example 3; FIG. 4B is a bar graph of the proportion of X sperm and Y sperm in the supernatant of the experimental group in Method 1 of Example 3; FIG. 4C shows the number of Y sperm and X sperm in the precipitate of the experimental group in Method 1 of Example 3; and FIG. 4D is a bar graph of the proportion of X sperm and Y sperm in the lower precipitate of the experimental group in Method 1 of Example 3.

FIG. 5 shows the experimental results of Method 2 in Example 3.

FIG. 6 shows the molecular structural formula of the 24e drug of the present invention.

FIG. 7 shows the changes in the straight-line velocity and average path velocity of the sex-sorted semen before and after elution. Figure is a statistical diagram of results of an anti-nuclease degradation experiment.

### DETAILED DESCRIPTION OF THE EMBODIMENTS

### Example 1: Effect of the Concentration of 24e Drug

The effect of the 24e drug on sperm was examined by incubating fresh bovine semen in a sperm sorting protection solution supplemented with different amounts of the 24e drug.

Fresh bovine semen provided by the Grassland Livestock Germplasm Innovation and Breeding Base of Inner Mongolia University was used. The fresh semen was diluted 15 times: 1 mL of fresh semen was taken and injected into 14 mL of in vitro fertilization solution kept at 37° C. The initial sperm motility and density were detected. The percentage of sperm with rapid and medium-speed movement was 92.7%, and the sperm density was 6x107 sperm/mL. The 24e drug was added to the sperm sorting protection solution at 37° C. to incubate bovine sperm for 60 min to prepare the sorting solution. The amounts of the 24e drug used were 0 μM, 0.15 μM, 0.21 μM, 0.27 μM, and 0.33 μM, respectively.

The in vitro fertilization solution was prepared as follows: each 1000 ml of the sperm sorting protection solution was prepared by mixing 6.902 g of sodium chloride, 0.300 g of potassium chloride, 0.330 g of calcium chloride dihydrate, 0.097 g of sodium dihydrogen phosphate, 2.500 g of glucose, 0.138 g of sodium pyruvate, 3.105 g of sodium bicarbonate, 2.5 mL of penicillin-streptomycin, 400 μL of phenol red, and deionized water.

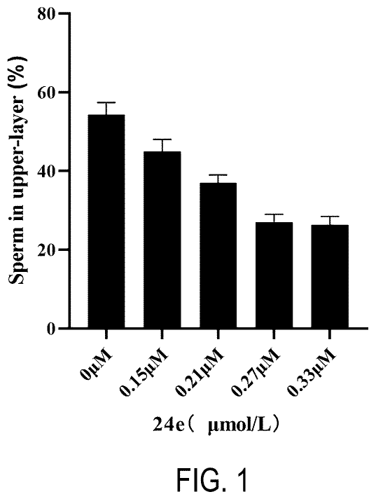

Real-time fluorescence quantitative PCR was used to verify the percentage of sperm floating in the upper layer after incubating bovine sperm. The results are shown in FIG. 1 and Table 1.

As can be seen from FIG. 1 and Table 1, the percentage of sperm floating in the upper layer gradually decreased with the increase in the amount of the 24e drug added. When the concentration of the 24e drug in the sorting solution was 0.27 μM-0.33 μM, the percentage of sperm in the upper layer decreased to approximately half of that in the group without the 24e drug added and tended to be stable. Since the 24e drug can specifically act on X sperm to inhibit the activity of X sperm, when the concentration of the 24e drug is 0.27 μM-0.33 μM, after the sperm is incubated at 37° C., the activity of X sperm is maximally inhibited and sinks to the lower layer, while the activity of Y sperm is not affected and tends to float upward. Studies have shown that the ratio of X sperm to Y sperm in the fresh semen of adult bulls is close to 1:1. Therefore, it is determined that the range of 0.27 μM-0.33 μM is the optimal concentration range for the 24e drug to sort bovine sperm.

TABLE 1
Changes in the Density of Sperm in the Upper Layer

| | Concentration of 24e Drug (mol/L) | | | | |
|---|---|---|---|---|---|
| | 0 | 0.15 | 0.21 | 0.27 | 0.33 |
| Density of Sperm in the | 60.15 | 50.18 | 41.62 | 30.36 | 29.87 |
| Upper Layer (million/mL) | | | | | |

### Example 2: Effect of the Concentration of Creatine

Based on the exploration in Example 1 that the range of 0.27 μM-0.33 μM is the optimal concentration range for the 24e drug to sort bovine sperm, the effect of creatine on the sperm sorting effect was examined by incubating bovine sperm with creatine added to the sorting solution with a 24e drug concentration of 0.3 μM.

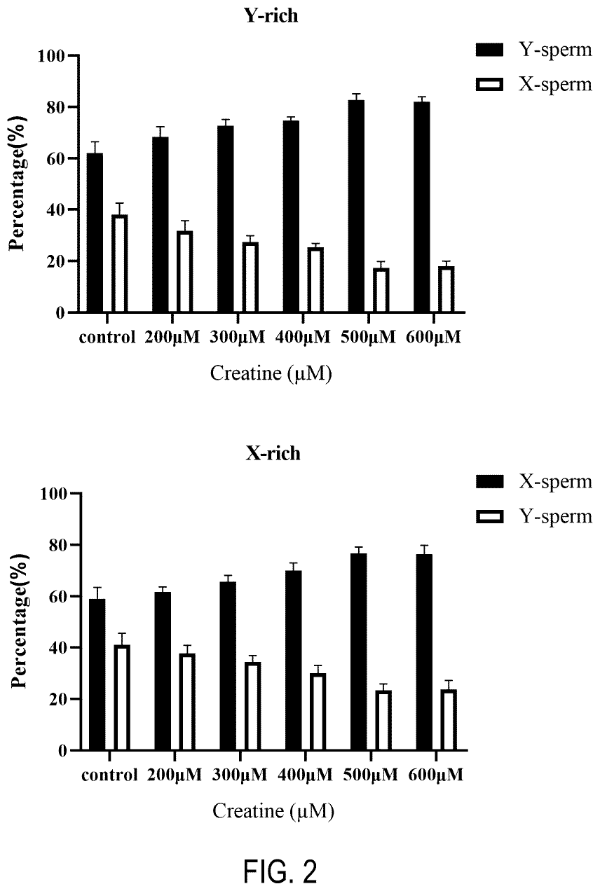

Fresh bovine semen provided by the Grassland Livestock Germplasm Innovation and Breeding Base of Inner Mongolia University was used, washed with in vitro fertilization solution, and after dilution, the sperm density was 6x107 sperm/mL. At 37° C., the 24e drug was added to the sperm sorting protection solution to make the final concentration of the 24e drug 0.3 μM. On this basis, creatine was added, and bovine sperm was incubated for 60 min. The amounts of creatine used were 0 μM (control group), 200 μM, 300 μM, 400 μM, 500 M, and 600 μM. The sorting effect of sperm at each creatine concentration was obtained by analyzing the sperm in the upper and lower layers, as shown in FIG. 2. As can be seen from FIG. 2, when the creatine concentration is 500 μM and 600 μM, the effect is the best and tends to be stable. When the creatine concentration is 500 μM, the percentage of X sperm in the lower layer of sorted sperm is 70.33%, and the percentage of Y sperm is 29.67%. In the upper layer of sperm, the percentage of X sperm is 22%, and the percentage of Y sperm is 78%. Creatine can enhance the activity of sperm, so that Y sperm floats up faster, while the activity of X sperm is inhibited by the 24e drug and sinks, making the stratification more obvious and improving the sorting efficiency. When the sperm density is 6x10\hat{ }7 sperm/mL, the concentration of the 24e drug is 0.27 μM-0.33 μM, and the creatine concentration is 500 μM-600 μM, the sorting effect on bovine sperm is the best.

### Example 3: Verification of the Sorting Effect of Bovine Semen

1. Preparation of Bovine Sex-Sorted Semen

(1) Fresh bull semen was collected to detect the vitality and density. The sperm vitality was detected using an animal semen analysis system, and the sperm density was detected using a sperm densitometer. The semen with a sperm vitality of more than 90% was selected as the semen to be sorted for subsequent sorting experiments.

Among them, the sperm densitometer is AccuCell; and sperm vitality refers to the ratio of the number of rapidly moving sperm and moderately moving sperm to the total number of sperm.

(2) The vitality of the fresh semen was 93%, and the density was 1.22x10⁸ sperm/mL.

2 mL of semen was taken and placed in a sorting container, and 2 mL of frozen semen diluent kept at 37° C. was added and mixed by inversion. Centrifugation was performed at 37° C. and 4000 rpm for 3 min. The upper layer of the frozen semen diluent and seminal plasma mixture was discarded, and the sperm pellet was retained. 4 mL of bovine sex-sorted sperm sorting solution kept at 37° C. was added to resuspend the sperm, and the sperm density was adjusted to 6×10⁷ sperm/mL.

Among them, the frozen semen diluent is the French CASA OptiXcell diluent; the volume of the bovine sex-sorted sperm sorting solution added is determined according to the density of the sperm to be sorted. The optimal concentration of the 24e drug when the sperm density is 6×10⁷ sperm/mL explored in Example 1, so adding the bovine sex-sorted sperm sorting solution to make the sperm density reach 6×10⁷ sperm/mL can achieve the best sorting effect.

(3) After static incubation at 37° C. for 60 min, the sperm floating in the upper layer was collected to obtain a sperm population rich in sperm carrying the Y chromosome. The sperm was collected using a pipette. The collected sperm population mainly contains sperm carrying the Y chromosome. Sperm carrying the Y chromosome fertilizes the egg to produce a fertilized egg with XY chromosomes. Therefore, using the collected sperm population for artificial insemination or in vitro fertilization can selectively produce male offspring.

On the other hand, the sperm population in the lower layer was collected to obtain a sperm population rich in sperm carrying the X chromosome. Sperm carrying the X chromosome fertilizes the egg to produce a fertilized egg with XX chromosomes. Therefore, using the collected sperm population for artificial insemination or in vitro fertilization can selectively produce female offspring.

Among them, the collected upper and lower layer semen should each account for 33.3% of the total volume (divided into three layers in total, so both the upper and lower layers are 33.3%). The upper layer is Y semen, and the lower layer is X semen.

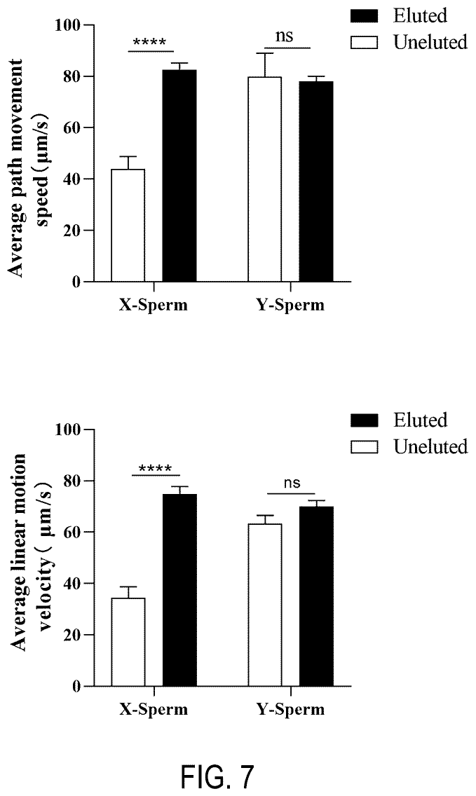

(4) The drugs in the X semen and Y semen were eluted and resuspended respectively using the sperm sorting protection solution (in actual operation, the frozen semen diluent can also be used) to obtain bovine sex-sorted semen (i.e., Y sex-sorted semen and X sex-sorted semen). The straight-line velocity and average path velocity of the sperm before and after elution are shown in FIG. 7. It can be seen from the figure that the vitality of X sperm after elution is increased by nearly 2 times, and the vitality of Y sperm is not significantly changed. The bovine sex-sorted sperm sorting solution does not significantly affect the vitality of bovine sperm.

2. Test of Sorting Effect of Bovine Sex-Sorted Semen

Method 1: Verification of Sorting Effect Using a Flow Cytometer

(1) Prepare the sorted bull X semen and Y semen as the experimental groups and the unsorted bull semen as the control group. Wash the sperm once with phosphate-buffered saline, centrifuge at 1500 rpm for 5 min to collect the sperm, detect the sperm density, and adjust the sperm density to a concentration of 1×106 sperm/ml. Take 1 ml of the sperm suspension, where the ratio of phosphate-buffered saline to semen volume is 1:1.

(2) After centrifuging the prepared sperm suspension, remove the supernatant, add 500 ul of 70% pre-cooled ethanol to the cells, and thoroughly pipette and mix evenly for fixation for 2 h to overnight, and store at 4° C.

(3) Add 100 μl of ribonuclease A solution to the cell pellet, resuspend the cells, and incubate in a 37° C. water bath for 30 min.

(4) Then add 400 μl of propidium iodide and mix evenly (wash away 70% ethanol with phosphate-buffered saline before staining), and filter once using a 200-mesh cell sieve to obtain a single-sperm suspension.

(5) Incubate at 4° C. in the dark for 30 min.

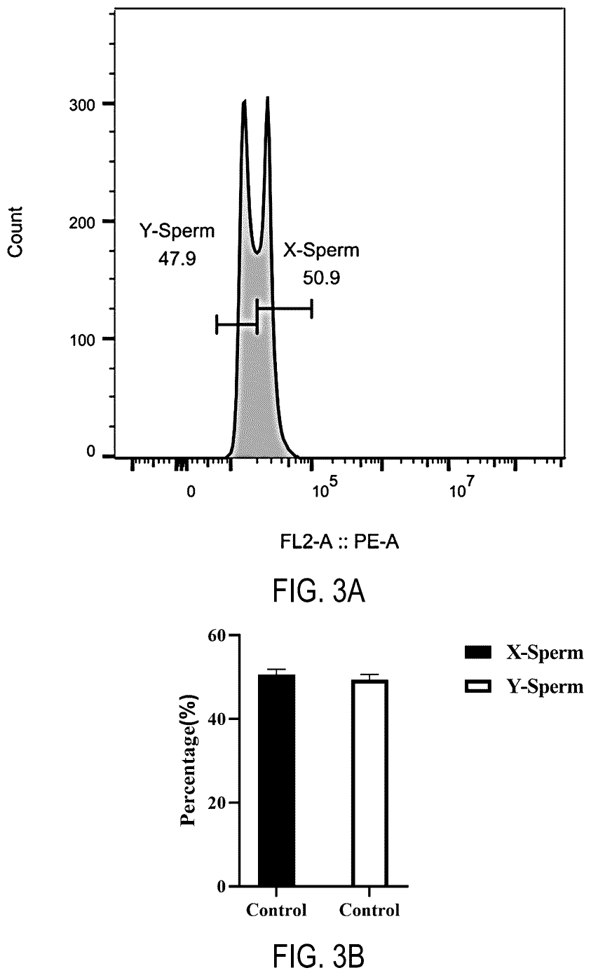

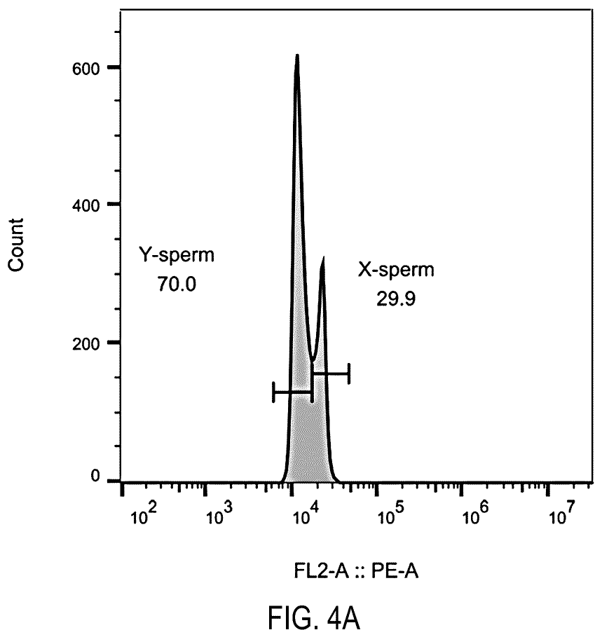

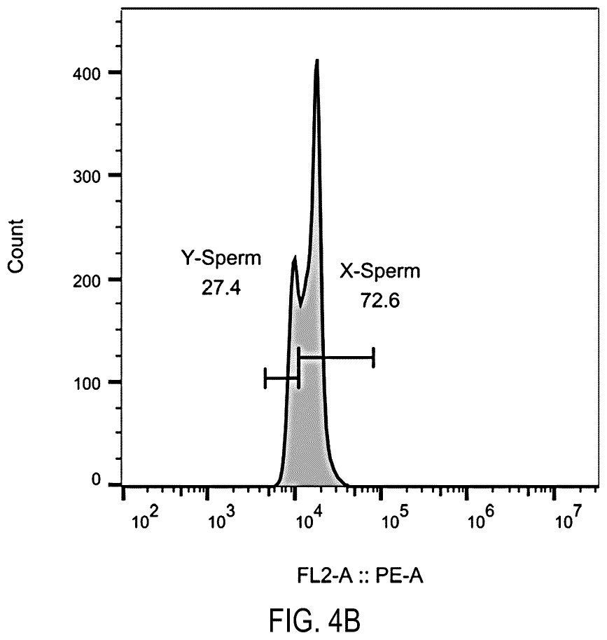

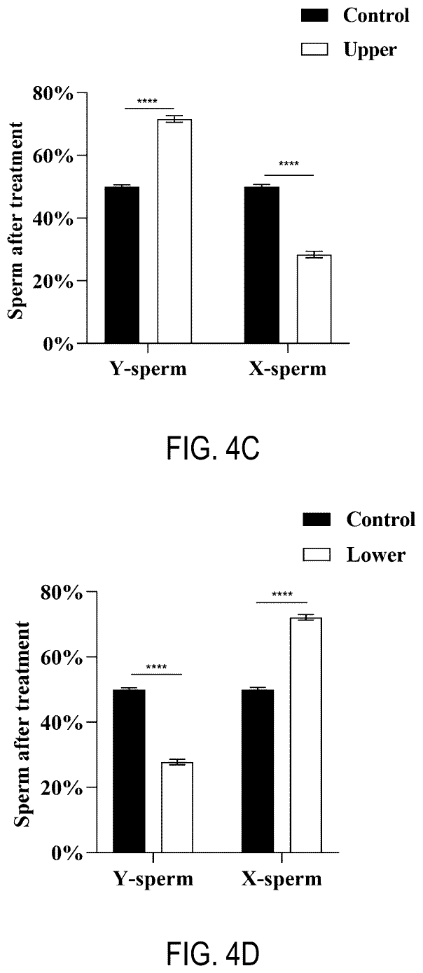

Machine Detection: Detect using a Cytoflex flow cytometer. The detection results are shown in FIGS. 3A to 3B and FIGS. 4A to 4D. FIG. 3A shows the number of X sperm and Y sperm in the control group, and FIG. 3B is a bar graph of the proportion of X sperm and Y sperm in the control group; and it can be seen from FIG. 3B that in the control group, the X sperm and Y sperm are 50.7% and 49.3%, respectively. The ratio of X sperm to Y sperm in the control group semen is in line with the ratio of X sperm to Y sperm in the semen before sorting, which is close to 1:1.

FIG. 4A shows the proportion of Y sperm and X sperm in the upper supernatant of the experimental group. FIG. 4C is a bar graph of the proportion of X sperm and Y sperm in the upper supernatant of the experimental group; and it can be seen from FIG. 4C that in the upper semen, the X sperm and Y sperm are 29.1% and 70.9%, respectively.

FIG. 4B shows the proportion of Y sperm and X sperm in the lower precipitate of the experimental group; FIG. 4D is a bar graph of the proportion of X sperm and Y sperm in the lower precipitate of the experimental group; and it can be seen from FIG. 4D that in the precipitate, the X sperm and Y sperm are 72.8% and 27.2%, respectively.

Method 2: Verification of Sorting Effect Using Real-time Fluorescence Quantitative PCR

(1) Primer Design

To verify the sorting effect, in this example, three pairs of primers were designed, namely the primer pairs for the Y chromosome-specific gene SRY, the X chromosome-specific gene PLP, and the internal reference gene GAPDH.

The Y chromosome-specific gene SRY gene primer pair is SRY-F and SRY-R.

The nucleotide sequence of SRY-F is as shown in SEQ ID NO.1; the nucleotide sequence of SRY-R is as shown in SEQ ID NO.2.

The X chromosome-specific gene PLP gene primer pair is PLP-F and PLP-R.

The nucleotide sequence of PLP-F is as shown in SEQ ID NO.3; the nucleotide sequence of PLP-R is as shown in SEQ ID NO.4.

The internal reference gene GAPDH primer pair is GAPDH-F and GAPDH-R.

The nucleotide sequence of GAPDH-F is as shown in SEQ ID NO.5; the nucleotide sequence of GAPDH-R is as shown in SEQ ID NO.6.

(2) Bovine Sperm DNA Extraction

The Solabio DNA extraction kit was used for sperm DNA extraction.

(a) Take 100 μl of semen into a 1.5 ml centrifuge tube, add 100 μl of Buffer GA solution, 20 μl of protein kinase, and 20 μl of dithiothreitol (with a concentration of 1 M), and shake vigorously to mix evenly for 15 sec.

(b) Incubate at 56° C. for 5 h, and shake thoroughly every 30 min.

(c) Centrifuge briefly for 10 sec, then add 200 μl of Buffer GB solution, shake vigorously to mix evenly for 15 sec, incubate at 70° C. for 10 min, centrifuge briefly for 10 sec, then add 200 μl of absolute ethanol, shake vigorously to mix evenly for 15 sec, and let stand at room temperature for 3 min, then centrifuge briefly for 10 sec.

(d) Transfer the lysed sperm into an adsorption column, centrifuge at 12000 rpm for 1 min, discard the waste liquid in the collection tube, then add 500 μl of Buffer GD solution, and centrifuge at 12000 rpm for 1 min, and discard the waste liquid in the collection tube.

(e) Add 600 μl of Buffer PW elution solution to the adsorption column and centrifuge at 12000 rpm for 1 min.

(f) Repeat step (5) once.

(g) Centrifuge at 12000 rpm in an empty state for 2 min, and let stand at room temperature for 5 min.

(h) Transfer the adsorption column into a new centrifuge tube, add 30 μl of deionized water to the adsorption column, let stand at room temperature for 5 min, and then centrifuge at 14000 rpm for 8 min to obtain the extracted bovine sperm DNA.

(3) DNA Concentration Detection: Use an ultra-micro spectrophotometer to detect the DNA concentration, and then dilute it to 100 ng/μl as the DNA quantitative template for real-time fluorescence quantitative PCR.

(4) Real-time Fluorescence Quantitative PCR

PCR system

| Reagent name | Volume (μl) |
|---|---|
| Real-time fluorescence quantitative | 10 |
| PCR enzyme | |
| Upstream primer-F (10 umol/L) | 0.4 |
| Downstream primer-R (10 umol/L) | 0.4 |
| Deionized water | 8.2 |
| Template DNA | 1 |
| Total volume | 20 |

qPCR procedure

| | | |
|---|---|---|
| Temperature | | |
| (° C.) | Time | Cycle |

| | | | |
|---|---|---|---|
| 95.0 | 30 | s | 1 |
| 95.0 | 10 | s | 40 |
| 60.0 | 30 | s | |
| 95.0 | 15 | s | 1 |
| 60.0 | 1 | min | 1 |
| 95.0 | 30 | s | 1 |
| 50.0 | 30 | s | 1 |

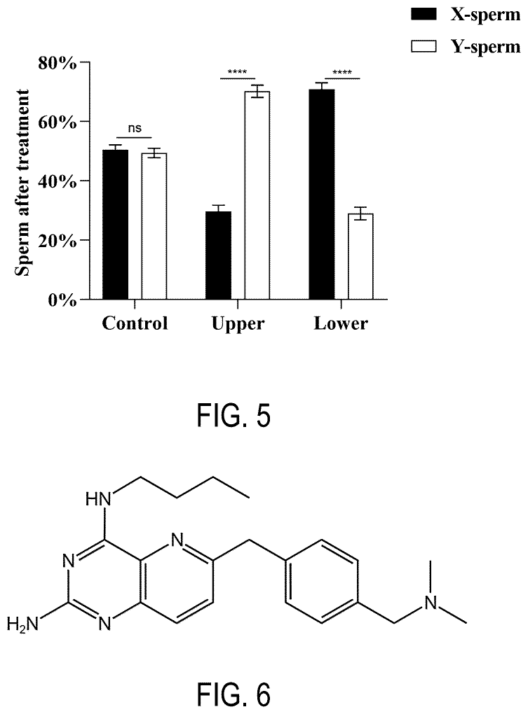

After collecting the sperm in the upper and lower layers respectively and extracting DNA, real-time fluorescence quantitative PCR uses the fluorescence after amplification of the specific genes in X and Y sperm to determine the percentages of X and Y sperm in the upper and lower layers. FIG. 5 shows the percentages of X and Y sperm in the upper and lower layer sperm. In the control group, X and Y sperm accounted for 51.9% and 48.1% respectively. In the precipitate, X and Y sperm accounted for 71.0% and 29.0% respectively. In the supernatant, X and Y sperm accounted for 29.8% and 70.2% respectively.

This example verified the sorting effect of X and Y sperm by two methods. The experimental data were basically consistent, proving that the sorting effect of this invention was accurate and reliable.

The above examples are only to illustrate the technical concept and characteristics of the present invention. The purpose is to enable those familiar with this technology to understand the content of the present invention and implement it accordingly, and it cannot limit the protection scope of the present invention. Any equivalent changes or modifications made according to the 5 essence of the present invention should be covered within the protection scope of the present invention.

What is claimed is:

1. A bovine sex-sorted sperm sorting solution mediated by a small molecule compound, comprising a sperm sorting protection solution and a sex control functional component, wherein the sperm sorting protection solution consists of sodium chloride, potassium chloride, calcium chloride dihydrate, sodium dihydrogen phosphate, glucose, sodium pyruvate, sodium bicarbonate, penicillin-streptomycin, and phenol red;
the sex control functional component comprises creatine and a 24e drug;
a concentration of the creatine is 450 μM-550 μM;
a concentration of the 24e drug is 0.27 μM-0.33 μM; and
the 24e drug is a pyrido[3,2-d]pyrimidine-based TLR7&8 dual agonist; a molecular formula of the 24e drug is C₂₁H₂₈N₆; and a molecular structure of the 24e drug is

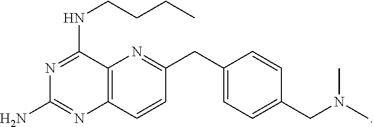 [SMILES: CCCCNc1nc(N)nc2ccc(Cc3ccc(CN(C)C)cc3)nc12]

2. The bovine sex-sorted sperm sorting solution mediated by the small molecule compound according to claim 1, wherein each 1000 ml of the sperm sorting protection solution is prepared by mixing 6.902 g of sodium chloride, 0.300 g of potassium chloride, 0.330 g of calcium chloride dihydrate, 0.097 g of sodium dihydrogen phosphate, 2.500 g of glucose, 0.138 g of sodium pyruvate, 3.105 g of sodium bicarbonate, 2.5 mL of penicillin-streptomycin, 400 μL of phenol red, and deionized water.

3. The bovine sex-sorted sperm sorting solution mediated by the small molecule compound according to claim 2, wherein 98.41 μg-120.28 μg of the 24e drug is added to each 1000 ml of the sperm sorting protection solution.

4. The bovine sex-sorted sperm sorting solution mediated by the small molecule compound according to claim 3, wherein 59.01 mg-72.12 mg of the creatine is added to each 1000 ml of the sperm sorting protection solution.

5. The bovine sex-sorted sperm sorting solution mediated by the small molecule compound according to claim 4, wherein a usage method is as follows:
(1) after detecting a vitality of collected fresh bovine semen, diluting the collected fresh bovine semen with the sperm sorting protection solution, followed by centrifuging, and after removing a supernatant of the bovine semen, resuspending the bovine semen with the bovine sex-sorted sperm sorting solution;
(2) incubating statically under a condition of 35° C.-40° C. for 60 min-90 min to obtain an incubated layered semen;
(3) separating the above layered semen to obtain a supernatant of the layered semen and a precipitate of the layered semen, wherein the supernatant of the layered semen is Y semen and the precipitate of the layered semen is X semen; and
(4) eluting the 24e drug in the Y semen and the X semen respectively with the sperm sorting protection solution to obtain a Y sex-sorted semen and an X sex-sorted semen for any one of in vitro fertilization, artificial insemination, and intracytoplasmic sperm injection.

6. Application of the bovine sex-sorted sperm sorting solution mediated by the small molecule compound according to claim 1, wherein the bovine sex-sorted sperm sorting solution mediated by the small molecule compound is used for identifying a type of bull sperm.

7. Application of the bovine sex-sorted sperm sorting solution mediated by the small molecule compound according to claim 1, wherein the bovine sex-sorted sperm sorting solution mediated by the small molecule compound is used for preparing a kit for identifying a type of bull sperm.

8. Application of the bovine sex-sorted sperm sorting solution mediated by the small molecule compound according to claim 1, wherein the bovine sex-sorted sperm sorting solution mediated by the small molecule compound is used for sorting or screening bull X and Y sperm.

9. Application of the bovine sex-sorted sperm sorting solution mediated by the small molecule compound according to claim 1, wherein the bovine sex-sorted sperm sorting solution mediated by the small molecule compound is used for preparing a kit for sorting or screening bull X and Y sperm.


SEQUENCE LISTING (6 sequences)

SEQ ID NO: 1 - DNA, 25 bp, synthetic construct
  source 1..25: mol_type = other DNA
gaaaataagcacaagaaagtccagg

SEQ ID NO: 2 - DNA, 21 bp, synthetic construct
  source 1..21: mol_type = other DNA
caaaaggagcatcacagcagc

SEQ ID NO: 3 - DNA, 20 bp, synthetic construct
  source 1..20: mol_type = other DNA
tgtgtggctccaaccttctg

SEQ ID NO: 4 - DNA, 20 bp, synthetic construct
  source 1..20: mol_type = other DNA
gctgtctccaagctatccca

SEQ ID NO: 5 - DNA, 20 bp, synthetic construct
  source 1..20: mol_type = other DNA
aagaggccactctgttaccc

SEQ ID NO: 6 - DNA, 20 bp, synthetic construct
  source 1..20: mol_type = other DNA
ttactagagggtgggcaagc